SEQUENCE LISTING (11 sequences)

SEQ ID NO: 1 - protein, 258 aa, Giardia lamblia
MSTWLLDSKCVERMKWLFSDLPEEKRVMIKMNEVAFFSVTPAVYADEVARMMRTVLALLGKPPYAVIDGTACVGGDTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 2 - protein, 258 aa, Giardia lamblia
  MISC_FEATURE (34)..(34): Xaa = any amino acid except valine (Val); preferably Ala, Gly, Met
MSTWLLDSKCVERMKWLFSDLPEEKRVMIKMNEXAFFSVTPAVYADEVARMMRTVLALLGKPPYAVIDGTACVGGXTRLLAKHFDMTVAIEXDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 3 - protein, 258 aa, Giardia lamblia
  MISC_FEATURE (34)..(34): Xaa = Ala, Gly or Met
MSTWLLDSKCVERMKWLFSDLPEEKRVMIKMNEXAFFSVTPAVYADEVARMMRTVLALLGKPPYAVIDGTACVGGDTRLLAKHFDMTVAIEXDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 4 - protein, 258 aa, Giardia lamblia
MSTWLLDSKCVERMKWLFSDLPEEKRVMIKMNEAAFFSVTPAVYADEVARMMRTVLALLGKPPYAVIDGTACVGGDTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 5 - protein, 258 aa, Giardia lamblia
MSTWLLDSKCVERMKWLFSDLPEEKRVMIKMNEGAFFSVTPAVYADEVARMMRTVLALLGKPPYAVIDGTACVGGDTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 6 - protein, 258 aa, Giardia lamblia
MSTWLLDSKCVERMKWLFSDLPEEKRVMIKMNEMAFFSVTPAVYADEVARMMRTVLALLGKPPYAVIDGTACVGGDTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 7 - protein, 258 aa, Giardia lamblia
MSTWLLDSKCVERMKWLFSDLPEEKRVMIKMNEAAFFSVTPAVYADEVARMMRTVLALLGKPPYAVIDGTACVGGTTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 8 - protein, 258 aa, Giardia lamblia
MSTWLLDSKCVERMKWLFSDLPEEKRVMIKMNEAAFFSVTPAVYADEVARMMRTVLALLGKPPYAVIDGTACVGGLTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 9 - protein, 258 aa, Giardia lamblia
MSTWLLDSKCVERMKWLFSDLPEEKRVMIKMNEAAFFSVTPAVYADEVARMMRTVLALLGKPPYAVIDGTACVGGVTRLLAKHFDMTVAIERDPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 10 - protein, 258 aa, Giardia lamblia
MSTWLLDSKCVERMKWLFSDLPEEKRVMIKMNEAAFFSVTPAVYADEVARMMRTVLALLGKPPYAVIDGTACVGGDTRLLAKHFDMTVAIEADPETYALLQDNLTTWGVDAKTISGDTAALIPQFWTLIGAVATFSLYLDPPWGGVDYRSQTDIQLTLGSLAVEDVVNRAFEAHLSMKLAVLKLPRNYNCGYLFRKLGKHEVFRITQGNFFVFFVARRGSRVKEHGRTAMLQLRKAREEAKARSEETKEDGETRGSGE

SEQ ID NO: 11 - protein, 243 aa, Giardia intestinalis
MSEWPRDKKCVERMKWLFSDLPEEERVKVKMNETAFFSVTPAVYAEKVAGMMRAVLALLGKSPYAVIDGTACVGGDTRLLAKHFDLTVAIEKDPETYTLLQDNLSTWGINVQTVSGDTSALIPQFWTLIGTVATFCLYLDPPWGGVDYRSQTDIQLTLGSLAVEDVINRAFEAHLSMKLAVLKLPRNYNCGYLFRKLGKHEVFRITQGNFFVFFVARHGSRVKEHCRAVLAQARKEKEAREGQ

(19) United States
(12) Patent Application Publication
(10) Pub. No.: US 2016/0032257 A1
(43) Pub. Date: Feb. 4, 2016

(54) AGENT AND METHOD FOR MODIFYING THE 5' CAP OF RNA

(71) Applicant: UNIVERSITÄT HAMBURG, Hamburg (DE)

(72) Inventors: Andrea Rentmeister, Muenster (DE); Daniela Stummer, Hamburg (DE)

(21) Appl. No.: 14/648,957

(22) PCT Filed: Dec. 6, 2013
(86) PCT No.: PCT/DE2013/200337

§ 371 Date: Jun. 2, 2015

(30) Foreign Application Priority Data
Dec. 10, 2012 (DE) 10 2012 222 675.3

(51) Int. Cl. C12N 9/10; C12P 19/32

(52) U.S. Cl. CPC C12N 9/1007; C12P 19/32; C12Y 201/01

(57) ABSTRACT

An agent and method for modifying the 5′ cap of RNA, for example for the purposes of isolation and analysis. According to one aspect the invention provides modified enzymes, namely modified trimethylguanosine synthases 2 from Giardia lamblia (GlaTGS2), the enzymatic activity of which is changed such that as compared to wild type enzymes the former can use AdoMet analogues better as cofactors.

The invention relates to an agent and method for modifying the 5′ cap of RNA.

Important information can be obtained about the state of a cell by examining its genotype, for example as to whether endogenous regulatory processes are proceeding correctly or whether changes compared with the normal state are present. This information can then, for example, be used to establish whether a cell is degenerate, infected with viruses or is in an irregular or diseased state. In this manner, for example, conclusions may be drawn as regards the presence of any diseases.

In this context, for example, it is known that the expression of genes can be investigated by isolation and analysis of the mRNA molecules present in the cells. In order to obtain reliable information, the quality of the mRNA preparation in this regard is of particular importance. Since cells contain many other molecules as well as other RNA species in addition to mRNAs, in particular non-coding RNAs (for example sRNAs, tRNA, rRNA, ncRNA, miRNA etc.), selective enrichment must be carried out on the basis of specific characteristics. In order to isolate mRNA specifically from eukaryotes, its characteristic features are exploited, namely the so-called poly(A) tail at the 3′ end and the cap structure at the 5′ end (J. Pease, R. Sooknanan, Nat Meth 2012, 9; J. S. Marcus, W. F. Anderson, S. R. Quake, Analytical Chemistry 2006, 78, 3084-3089; Z. Y. Yang, H. J. Edenberg, R. L. Davis, Nucleic Acids Res 2005, 33; M. E. Folkers, D. A. Delker, C. I. Maxwell, C. A. Nelson, J. J. Schwartz, D. A. Nix, C. H. Hagedorn, Plos One 2011, 6; Z. Gao, Q. Zhang, Y. Cao, P. Pan, F. Bai, G. Bai, Journal of Chromatography A 2009, 1216, 7670-7676; U. Schibler, D. Rifat, D. J. Lavery, Methods 2001, 24, 3-14; E. Z. Bajak, C. H. Hagedorn, Methods in molecular biology (Clifton, N.J.) 2008, 419, 147-160; A. K. Shukla, A. K. Shasany, S. P. S. Khanuja, Indian journal of experimental biology 2005, 43, 197-201). The mRNA is normally non-covalently bonded to other molecules via these features which, for example, are immobilized on appropriate column materials (for example binding via the poly(A) tail to oligo-dT-columns or via the 5′ cap to the protein eIF4E).

Currently, the poly(A) tail contained in mRNAs is the primary isolation tool. Like the cap, this is a characteristic structure in mature mRNA molecules and miRNA precursors (pri-miRNA). The molecules can hybridize via this region onto immobilized complementary deoxynucleotide (oligo-dT) probes and thus be isolated from complex samples. The column materials used up until now (beads) are known in the art and are industry standards.

Bajak and Hagedorn (E. Z. Bajak, C. H. Hagedorn, Methods in molecular biology (Clifton, N.J.) 2008, 419, 147-160) have established a method in which a variant of the translation initiation factor eIF4E is used to isolate RNA via its cap structure (see also U.S. Pat. No. 6,841,363 B2, Gowda, Nucleic Acids Research, 2010, 38, 21, 7558-7569). One advantage in this regard is that RNA molecules are identified and enriched independently of the length of their poly(A) tail, solely on the basis of the cap structure they contain, whereupon RNAs with a short poly(A) tail (both mRNAs and miRNA precursors) are also accessible for subsequent analyses. By carrying out the assay, an eIF4E variant which has an up to 10-fold higher affinity for the target molecules than the wild type protein, is immobilized via a protein affinity tag (in particular glutathione S transferase, GST) on glutathione beads and incubated with the sample. The RNA with a cap structure binds non-covalently to the immobilized protein and thus can be isolated from the complex sample with the aid of the beads. In this manner, RNA molecules of hepatitis C-infected cells could be isolated independently of the varying motif of the poly(A) tail. By carrying out a “next generation sequencing” experiment, new predictions regarding the changes in gene regulation in the host cell after infection by the virus could be made (M. Folkers, PLOS one, 2011, 6, 2, e14697, Papic, 2012, Viruses, 2012, 4, 581.612).

One disadvantage of that method is the lack of opportunity for covalently binding RNA molecules directly to a support, whereupon selection of the washing conditions when separating from the impurities is restricted. Another restriction to carrying out this method is the frequent 1:1 relationship between the binder molecule and the RNA molecule.

Dalhoff et al. (Dalhoff C, Lukinavicius G, Klimas\check{a}uskas S, Weinhold E, 2006, Nat Chem Biol. 2(1): 31-32) describe the direct transfer of an ethyl, propyl, propenyl and 2-butynyl group onto 2′-deoxycytidine and 2′-deoxyadenosine by three S-adenosyl-L-methionine(AdoMet)-dependent DNA methyl transferases using appropriate analogues of this co-factor. In this regard, the AdoMet analogue carries the appropriate group on the sulphur atom instead of a methyl group. Lukinavi\check{c}ius et al. (G. Lukinavi\check{c}ius, V. Lapienė, Z. Sta\check{s}evskij, C. Dalhoff, E. Weinhold, S. Klima\check{s}auskas, J. Am. Chem. Soc. 2007, 129, 2758-2759) used a further AdoMet analogue containing a NH₂ group to transfer this group to DNA nucleosides by means of suitable DNA methyl transferases. Motorin et al also had a similar approach when they described the use of a combination of enzymatic transfer and click chemistry for site-specific labelling of tRNA molecules for biophysical studies (Y. Motorin, J. Burhenne, R. Teimer, K. Koynov, S. Willnow, E. Weinhold, M. Helm, Nucleic Acids Research, 2010, 1-10, doi: 10.1093/nar/gkq825). Here, AdoEnYn, also an analogue of the co-substrate S-adenosyl-L-methionine, was used to enzymatically transfer the pentenene residue CH≡C—CH═CH—CH₂— by means of tRNA:methyl transferase Trml onto the exocyclic N2 atom of the guanosine in position 26 of a tRNA^{phe}. Next, a fluorophore was bound to the modified tRNA^{phe} by means of a Cu(I)-catalysed Azide Alkyne 1,3-dipolar cycloaddition (CuAAC). In addition, sequence-specific click labelling of RNA was obtained using box C/D RNP methyltransferases (M. Tomkuvienė, B. Clouet-d'Orval, I. \check{C}erniauskas, E. Weinhold, S. Klima\check{s}auskas, Nucleic Acids Res 2012).

However, there is still a need for further opportunities for isolating specific RNA molecules, in particular mRNA molecules, from cells and for carrying out an analysis.

Thus, the aim of the present invention is to provide such an opportunity.

This aim is accomplished by the subject matter of the independent claims below. Appropriate embodiments of the invention are provided in the dependent claims.

It has surprisingly been observed that an enzyme which is modified at a specific position, namely the trimethylguanosine synthase 2 from Giardia lamblia (hereinafter abbreviated to “GlaTgs2”), the wild type sequence of which is provided in SEQ ID NO: 1, provides novel possibilities for labelling and/or isolation of RNA species which have a 5′-m⁷GpppN cap. This m⁷GpppN cap is a guanosine residue which is methylated in the N7 position which is bonded via a triphosphate ester bridge to the 5′ end of a RNA molecule (5′-5′ linkage), as can be seen from the following formula (II):

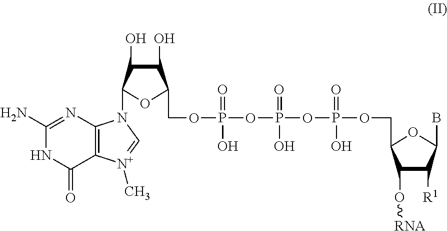

in which R¹ is OH or OCH₃. The B in the above formula represents any nucleobase. The N in the term m⁷GpppN represents a nucleoside, nucleotide, nucleoside analogue or nucleotide analogue, ppp represents the triphosphate bridge, G represents guanosine and m⁷ represents the methyl group at N7.

Eukaryotic mRNA molecules, for example, have such a cap, and also specific non-coding RNA species, for example snRNAs, snoRNAs and telomerase-RNAs.

Wild type GlaTgs2 (see SEQ ID NO: 1) has 258 amino acids and catalyses the further methylation (hypermethylation) of the cap guanosine at the N2 position with S-adenosyl-L-methionine (AdoMet) as a co-factor (S. Hausmann et al, J. Biol. Chem. 2008, 283, 31706-31718). In contrast to the human trimethylguanosine synthase hTgs, which can catalyse the transfer of two methyl residues to N2, the enzyme from Giardia lamblia does not appear to accept any dimethylated nucleotides as a substrate, so that only a single methyl residue can be transferred onto the N2 with this enzyme. Thus, here we should actually have a dimethylguanosine synthase and not a trimethylguanosine synthase. In order to avoid misunderstandings, however, the term trimethylguanosine synthase, abbreviated to Tgs, will be used for this enzyme.

AdoMet is also abbreviated to “SAM” and acts with various enzymes as a co-factor for the transfer of the methyl group on the sulphur atom. After cleavage of the CH₃ group, S-adenosyl-L-homocysteine remains behind; this is also abbreviated to “AdoHcy” or “SAH”.

It has now surprisingly been found that an amino acid exchange at position 34 of the wild type GlaTgs2 of SEQ ID NO: 1, which results in exchanging the amino acid valine at this position for another amino acid, preferably a non-polar/hydrophobic or polar/neutral amino acid, and particularly preferably for alanine, glycine or methionine, substantially changes the activity of the resulting enzyme in a manner such that it can use AdoMet analogues as co-factors or can exploit them better compared with the wild type enzyme. Preferably, the amino acid introduced in place of valine is not tryptophan or leucine. This opens up the possibility of a targeted modification of the 5′ end of RNA species which carry an m⁷GpppN cap so that in subsequent steps, reporter groups can be introduced and/or a specific immobilization of this RNA species can be obtained.

Examples of non-polar/hydrophobic amino acids are alanine, valine, methionine, leucine, isoleucine, proline, tryptophan and phenylalanine. Examples of polar/neutral amino acids are tyrosine, threonine, glutamine, glycine, serine, cysteine and asparagine.

The term “AdoMet analogue” as used here should be understood to mean a compound with the following formula:

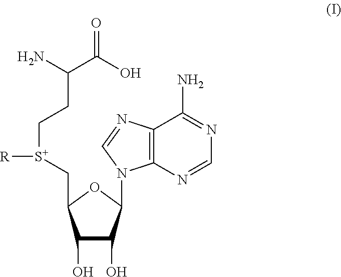

which carries a residue R on the sulphur atom which is not a methyl group. Thus, it is a compound with the same basic framework as AdoMet (S-adenosyl-L-methionine), wherein instead of the methyl group, another residue is bonded to the sulphur atom. Examples of AdoMet analogues are described in WO 2006/108678 A2.

An example of a residue of this type is propenyl, CH₂═CH—CH₂—. The corresponding AdoMet analogue (5′-[(S)-[(3S)-3-amino-3-carboxypropyl]prop-2-enylsulphonio]-5′-deoxyadenosine) with this residue instead of methyl is denoted “AdoPropen” and has the following formula (Ia):

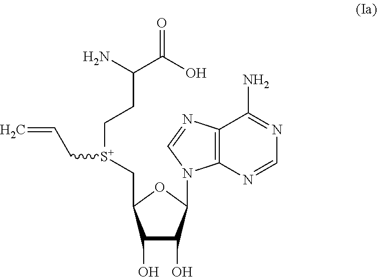

Other examples of the residue R are propynyl CH≡C—CH₂—, butynyl CH═C—CH₂—CH₂—, pent-2-en-4-ynyl CH≡C—CH═CH—CH₂—, benzyl Ph-CH₂-(Ph=C₆H₅) and azidobutenyl N₃—CH₂—CH═CH—CH₂—. In the case of the propynyl residue CH≡C—CH₂—, the AdoMet analogue is denoted here as “AdoPropin”; in the case of the butynyl residue CH≡C—CH₂—CH₂—, it is “AdoButin”; in the case of the pent-2-en-4-ynyl residue CH≡C—CH═CH—CH₂— it is “AdoEnYn”; in the case of the benzyl residue as “AdoBenzyl” and in the case of the azidobutenyl residue N₃—CH₂—CH═CH—CH₂— it is “AdoAzid”. The pent-2-en-4-ynyl residue will also be denoted here as the “pentenynyl residue.

Thus, in a first aspect, the invention provides an isolated or synthetic protein which:

a. is composed of or comprises an amino acid sequence in accordance with SEQ ID NO: 2, or
b. is composed of or comprises an amino acid sequence which is homologous with the amino acid sequence in accordance with SEQ ID NO: 2, with the proviso that in the homologous amino acid sequence, the amino acid at the position which corresponds to position 34 of SEQ ID NO: 2 is not valine, or
c. is composed of or comprises an amino acid sequence which is homologous with the amino acid sequence in accordance with SEQ ID NO: 2, wherein the amino acid sequence has more than 85%, preferably at least 90%, particularly preferably at least 95% identity with the amino acid sequence in accordance with SEQ ID NO: 2, with the proviso that in the homologous amino acid sequence the amino acid at the position which corresponds to position 34 of SEQ ID NO: 2 is not valine, or
d. is composed of or comprises a coherent partial sequence of at least 10, 15, 20, 25, 30, 35, 40, 45, 50, 60, 65, 70, 80, 90 or at least 100 amino acids, preferably at least 110, 120, 130, 140, 150, 160, 170, 180, 190 or at least 200 amino acids, particularly preferably at least 210, 220, 230, 240 or at least 250 amino acids of the amino acid sequence of a, b or c, with the proviso that the partial sequence comprises the amino acid at position 34 of SEQ ID NO: 2 or the corresponding homologous amino acid, and
e. is not composed of the amino acid sequence in accordance with SEQ ID NO: 11 (GL50581 2635 from Giardia intestinalis ATCC 50581, GenBank-Nr. EET00120.1).

The expression that the protein “is composed of an amino acid sequence” means that the protein consists of the sequence, i.e. no more amino acids are present at the C- and/or N-ends. The expression that the protein “comprises an amino acid sequence” means that the protein contains the sequence, but this is not limited to proteins having no other amino acids at the C- and/or N-end; however, this term also encompasses the expression that the protein “is composed of” an amino acid sequence, i.e. consists only of the amino acids set out in the sequence and in the order given in the sequence.

The term “protein” as used here denotes polymers formed from any number of amino acids which are connected together via peptide linkages and comprises the terms “peptide” and “polypeptide”. The linear succession of amino acids in a protein is denoted the “amino acid sequence”.

The term “synthetic” as used here means “produced artificially” and encompasses proteins which are not present in nature with that respective amino acid sequence. “Isolated” as used here means that a protein has been removed from its original or natural environment, for example from a eukaryotic or prokaryotic cell.

The tem “homologous” in relation to a protein means that the amino acid sequence of a protein is substantially identical to that of another protein with which it is being compared, without it being completely identical therewith. As an example, “homologous” may mean that a protein exhibits an identical amino acid sequence with the trimethylguanosine synthase of Giardia lamblia with the exception of one amino acid. The presence of a homology between two proteins can be established by comparing a respective position in one sequence with the corresponding position in the other sequence and determining whether identical or similar residues are present. Two mutually compared sequences are homologous when a specific minimum fraction of identical or similar amino acids are present. “Identity” means that, when comparing two sequences at equivalent locations, the same amino acid is present. In this regard, it may on occasion be necessary to allow for gaps in the sequence in order to obtain the best alignment of the compared sequences. “Similar amino acids” here are amino acids with the same or equivalent physico-chemical properties. Exchange of one amino acid by another amino acid with the same or equivalent physico-chemical properties is known as “conservative exchange”. Examples of physico-chemical properties of an amino acid are hydrophobicity or charge. In particular, the computer program “Basic Local Alignment Search Tool”, abbreviated to BLAST, (S. F. Altschul et al. (1990), Basic Local Alignment search tool, J. Mol. Biol. 215: 403-410; see, for example, http://www.ncbi.nlm.nih.gov/BLAST/), which uses the BLOSUM62 substitution matrix (Henikoff, S., and Henikoff, J., amino acid substitution matrices from protein blocks. Proc. Natl. Acad. Sci. USA. 89: 10915-10919, 1992) identifies as similar amino acids those non-identical amino acids which are assigned a positive point score in the BLOSUM62 substitution matrix. For the purposes of the present invention, a homology is acknowledged as being present between two sequences when an identity or similarity (positive), preferably identity, of at least 45%, preferably at least 50%, at least 55%, at least 60%, at least 65%, at least 70%, at least 75%, at least 80%, at least 85%, at least 90%, at least 95%, at least 97%, at least 98%, or at least 99% is obtained using the computer program BLAST (S. F. Altschul et al. (1990), Basic Local Alignment search tool, J. Mol. Biol. 215: 403-410; see, for example, http://www.ncbi.nlm.nih.gov/BLAST/) using standard default parameters (“Expect Threshold”=10, “Word size”=3, “Existence Gap Costs”=11, “Extension Gap Costs=1) and the BLOSUM62 substitution matrix (Henikoff, S., and Henikoff, J., amino acid substitution matrices from protein blocks. Proc. Natl. Acad. Sci. USA. 89: 10915-10919, 1992). Preferably, the starting length is a minimum length of 20, preferably a minimum length of 25, 30, 35, 40, 45, 50, 60, 80 or 100, more preferably a minimum length of 120, 140, 160, 180 or 200 amino acids, or a minimum length of 25%, 30%, 40%, 50%, 60%, 70%, 80%, 90% or 95% of the amino acids of the respective amino acid sequences. Particularly preferably, the starting length is the complete length of the respective protein. It would be obvious to the skilled person on the basis of his specialist knowledge which of the available BLAST programs, for example BLASTp, would be used to determine the homology. Furthermore, other programs exist which are known to the skilled person which he could, if necessary, draw upon to assess the homology of two or more of the sequences to be compared. Examples of such programs are available from the website of the European Bioinformatics Institute (EMBL) (see, for example, http://www.ebi.ac.uk/Tools/similarity.html). In particular, the term “homologous” as used in the present application means agreement, i.e. identity in the amino acid sequence, of at least 60%, preferably at least 65%, 70%, 75%, 80%, 85%, 90%, 91%, 92%, 93%, 94%, 95%, 96%, 97%, 98% or at least 99%, particularly preferably at least 99.5%. In particular, “homologous” can also mean that when compared with another protein, a trimethylguanosine synthase exhibits another, a missing or an additional amino acid at no more than 60, preferably no more than 50, 40, 30, 25, 20, 19, 18, 17, 16, 15, 14, 13, 12 or 11, particularly preferably no more than 9, 8, 7, 6, 5, 4, 3, 2 or 1 position(s).

The term “alkyl” encompasses saturated aliphatic (non-aromatic) groups, including straight-chain alkyl groups (for example methyl, ethyl, propyl, butyl, pentyl, hexyl, heptyl and octyl) and branched-chain alkyl groups (for example isopropyl, tert-butyl, isobutyl). The term also encompasses O-, N-, S- or P-alkyl groups (for example —O-methyl), i.e. alkyl groups which are bonded to a compound via an oxygen, nitrogen, sulphur or phosphorus atom.

The expression “Cₙ-Cₘ”, wherein n and m are each positive whole numbers and m is larger than n, signifies a range which gives the number of C atoms of a compound or a residue. The expression here expressly includes all integral intermediate numbers between the limits n and m, respectively independently of each other. The expression “C₁-C₁₀” (n=1, m=10), thus means a compound, a group or a residue containing 1-10, i.e. 1, 2, 3, 4, 5, 6, 7, 8, 9 or 10 C atoms. Thus, “C₁-C₁₀” at the same time includes, for example, “C₂-C₆”, i.e. 2, 3, 4, 5 or 6 C atoms, or “C₁-C₄”, i.e. 1, 2, 3 or 4 C atoms, or “C₄-C₉”, i.e. 4, 5, 6, 7, 8 or 9 C atoms. Similarly, the expression “C₂-C₁₀ alkyl”, for example, means an alkyl group containing 2, 3, 4, 5, 6, 7, 8, 9 or 10 C atoms and includes all combinations of values of n and m in the range from n=2 to m=10; for example, “C₅-C₇ alkyl” means an alkyl containing 5, 6 or 7 C atoms.

The expression “alkenyl” encompasses unsaturated aliphatic (non-aromatic) groups with at least one C—C double bond, including straight-chain and branched-chain alkenyl groups. The expression also encompasses O-, N-, S- or P-alkenyl groups (for example —O-propenyl), i.e. alkenyl groups which are bonded to a compound via an oxygen, nitrogen, sulphur or phosphorus atom. The expression “C₂-C₁₀ alkenyl” means an alkenyl group containing 2, 3, 4, 5, 6, 7, 8, 9 or 10 C atoms.

The expression “alkynyl” encompasses unsaturated aliphatic (non-aromatic) groups with at least one C—C triple bond, including straight-chain and branched-chain alkynyl groups. The expression also encompasses O-, N-, S- or P-alkynyl groups (for example —O-butynyl), i.e. alkynyl groups which are bonded to a compound via an oxygen, nitrogen, sulphur or phosphorus atom. The expression “C₂-C₁₀ alkynyl” means an alkynyl group containing 2, 3, 4, 5, 6, 7, 8, 9 or 10 C atoms.

The expression “alkenynyl” encompasses unsaturated aliphatic (non-aromatic) groups with at least one C—C double bond and at least one C—C triple bond, including straight-chain and branched-chain alkenynyl groups. The expression also encompasses O-, N-, S- or P-alkenynyl groups, i.e. alkenynyl groups which are bonded to a compound via an oxygen, nitrogen, sulphur or phosphorus atom. The expression “C₄-C₁₀ alkenynyl” means an alkenynyl group containing 4, 5, 6, 7, 8, 9 or 10 C atoms.

The expression “cycloalkyl” encompasses alicyclic groups, i.e. cyclic saturated aliphatic (non-aromatic) groups, for example cyclopropyl, cyclopentyl, cyclohexyl, cycloheptyl, cyclooctyl. The expression also encompasses O-, N-, S- or P-cycloalkyl groups, i.e. cycloalkyl groups which are bonded to a compound via an oxygen, nitrogen, sulphur or phosphorus atom. The expressions “cycloalkenyl”, “cycloalkynyl” and “cycloalkenynyl” respectively mean cyclic aliphatic (non-aromatic) alkenyl, alkynyl or alkenynyl as defined above, wherein the double and/or triple bond(s) may be present within or outside the ring or ring system.

The expression “heteroalkyl” denotes alkyl groups in which one or more carbon atoms of the hydrocarbon backbone have been replaced by other atoms (heteroatoms), for example oxygen, nitrogen, sulphur or phosphorus atoms. The expression also encompasses O-, N-, S- or P-heteroalkyl groups, i.e. heteroalkyl groups which are bonded to a compound via an oxygen, nitrogen, sulphur or phosphorus atom. The expression “heteroalkyl” also encompassed cycloalkyls in which one or more carbon atoms of the hydrocarbon backbone are replaced by other atoms, for example oxygen, nitrogen, sulphur or phosphorus atoms. The expressions “heteroalkenyl”, “heteroalkynyl”, “heteroalkenynyl” should be understood to include alkenyls, alkynyls and alkenynyls as well as cycloalkenyls, cycloalkynyls and cycloalkenynyls in which one or more carbon atoms of the hydrocarbon backbone has been replaced by other atoms (heteroatoms), for example oxygen, nitrogen, sulphur or phosphorus atoms. The expression “C₁-C₁₀ heteroalkyl” means an alkyl group containing 1, 2, 3, 4, 5, 6, 7, 8, 9 or 10 C atoms and at least one heteroatom. This is also the case for heteroalkenyls, heteroalkynyls and heteroalkenynyls.

The term “azidoalkyl” as used here means an alkyl with an azido group, —N₃. The expression also includes cyclic alkyls and heteroalkyls with an azido group. The terms “azidoalkenyl”, “azidoalkynyl” and “azidoalkenynyl” should thus be understood to include alkenyls, alkynyls and alkenynyls, cyclic alkenyls, alkynyls and alkenynyls as well as hetero-alkenyls, -alkynyls and -alkenynyls.

The expression “substituted” means that one or more substituents are present which replace a hydrogen atom on one or more carbon atoms of the hydrocarbon backbone. Examples of substituents of this type are oxo, hydroxyl, phosphate, cyano and amino groups, but also, for example, halogens, (for example F, Cl, Br, I), alkyl, cycloalkyl, aryl and heteroaryl.

A “nucleic acid” should be understood to mean a polymer the monomers of which are nucleotides. A nucleotide is a compound formed from a sugar residue, a nitrogen-containing heterocyclic organic base (nucleotide or nucleobase) and a phosphate group. As a rule, the sugar base is a pentose; in the case of DNA, it is deoxyribose; in the case of RNA, it is ribose. The nucleotides are linked via a phosphate group by means of a phosphodiester bridge, generally between the 3′ C atom of the sugar component of a nucleoside (compound formed from a nucleobase and sugar) and the 5′ C atom of the sugar component of the next nucleoside. The expression “nucleic acid” as used here encompasses DNA, RNA and mixed DNA/RNA sequences, for example.

The term “nucleobase” should be understood to mean organic bases which occur in RNA or DNA. Nucleobases are often purines (R) and pyrimidines (Y). Examples of purines are guanine (G) and adenine (A); examples of pyrimidines are cytosine (C), thymine (T) and uracil (U). Phosphorylated nucleosides, for example nucleoside monophosphate (NMP), nucleoside diphosphate (NDP) and nucleoside triphosphate (NTP), are also described as nucleotides. The phosphate, diphosphate (pyrophosphate-) or triphosphate group is usually bonded to the 5′ C atom of the sugar component of the nucleoside, but may also be bonded to the 3′ C atom, for example.

The term “nucleoside” as used here should be understood to mean organic molecules which consist of a sugar residue (sugar component) and an organic base (base component), for example a heterocyclic organic base, in particular a nitrogen-containing heterocyclic organic base, which is bonded via a glycosidic linkage. The sugar residue is often a pentose, for example deoxyribose or ribose, but may also be another sugar, for example a C₃, C₄ or C₆ sugar. In particular, the term “nucleoside” should therefore be understood to mean a compound with general formula (III):

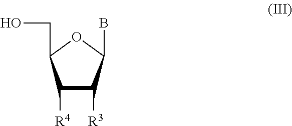

in which B is a nitrogen-containing heterocyclic organic base, for example a nucleobase, and R³ and R⁴ are independently H or OH.

The term “nucleoside analogue” as used here should be understood to mean a compound which is not naturally present in the human body, but is structurally similar to a nucleoside present in the human body so that, for example, it can be processed in the cell and/or by viral enzymes in a similar manner to the natural nucleoside, for example phosphorylated and incorporated into a RNA or DNA strand. A nucleoside analogue may itself be a nucleoside. However, it may also, for example, be another compound with the above properties, for example a compound formed from a heterocyclic base and an acyclic residue and/or from a residue which is not a sugar or a compound formed from a carbocyclic compound and a sugar residue. Nucleoside analogues are either themselves nucleosides in the above sense or structurally and/or functionally analogous to nucleosides. Since nucleoside analogues do not necessarily have to contain a sugar or base component in the strict sense, here again, the terms “base component-analogous components” (base analogue) or “sugar component-analogous component” (sugar analogue) are used. When the terms “sugar component” or “base component” are used, here, the appropriate analogous components are included, unless the context clearly indicates otherwise. Examples of nucleoside analogues are, for example, AZT (3′-azido-2′,3′-dideoxythymidine, azidothymidine), 2′,3′-dideoxyinosine (didanosine), 2′,3′-dideoxycytidine (zalcitabine) and 2-amino-9-((2-hydroxyethoxy)methyl)-1H-purine-6(9H)-one (acyclovir). Nucleoside phosphonates may also be nucleoside analogues.

The term “nucleotide” as used here means phosphorylated nucleosides, for example nucleoside monophosphate (NMP), nucleoside diphosphate (NDP) and nucleoside triphosphate (NTP). The phosphate, diphosphate (pyrophosphate) or triphosphate group is generally bonded with the 5′ C atom of the sugar component of the nucleoside, but may also be bonded with the 3′ C atom, for example. The term “nucleotide analogue” should thus also be understood to mean a phosphorylated nucleoside analogue.

The term “Total Turnover Number”, TTN, should be understood to mean the number of moles of product which is formed per mol of co-factor or enzyme over the whole reaction period.

The protein in accordance with the invention preferably enzymatically catalyses transfer of the residue R of the compound with the following formula (I):

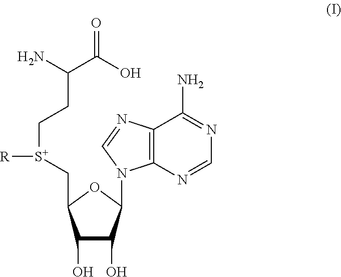

to the N2 of the guanosine of m⁷GTP, m⁷GpppN or a compound with the following formula (II):

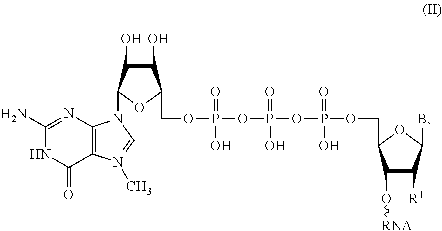

wherein RNA means ribonucleic acid, R¹ means OH or OCH₃, N means nucleoside, nucleotide, nucleoside or nucleotide analogue, B stands for nucleobase, and R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄₋₁₀ alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted C₁₋₁₀ azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl, propenyl CH₂═CH—CH₂—, propynyl C≡C—CH₂—, butynyl CH≡C—CH₂—CH₂—, pentenynyl CH≡C—CH═CH—CH₂— and azidobutenyl N₃—CH₂—CH═CH—CH₂—. In the case of benzyl, a substitution in the para-position is preferred, particularly preferably a substitution with an alkene, alkyne or azide.

The fact that the protein of the invention catalyses the transfer of the residue R of the AdoMet analogue of formula (I) does not mean that AdoMet cannot also be used as a co-factor and transfer a methyl group onto the N2 of the m⁷GpppN cap. Rather, this means that the reaction with the AdoMet analogue under physiological conditions is also at least catalysed by the protein of the invention, preferably at a higher rate compared with the wild type enzyme, with a greater affinity for the co-factor, higher yield and/or higher total turnover number (TTN).

Preferably, the total turnover number TTN of the protein of the invention is larger than 5, preferably ≧6, ≧7, ≧8, ≧9 or ≧10, and/or the total turnover number TTN of the protein of the invention is at least twice the total turnover number of the wild type enzyme, each with respect to the mean and to the same AdoMet analogue, preferably AdoPropen. More preferably, in the protein of the invention, the ratio of the total turnover number of AdoMet to AdoPropen, i.e. the ratio TTN_{Adomet}:TTN_{Adopropen}, is ≦20, particularly preferably 15, more preferably ≦14, ≦13, ≦12, ≦11 or ≦10.

The transfer of a residue R of an S-adenosyl-L-methionine analogue to an mRNA catalysed by the protein of the invention is illustrated in the scheme provided below:

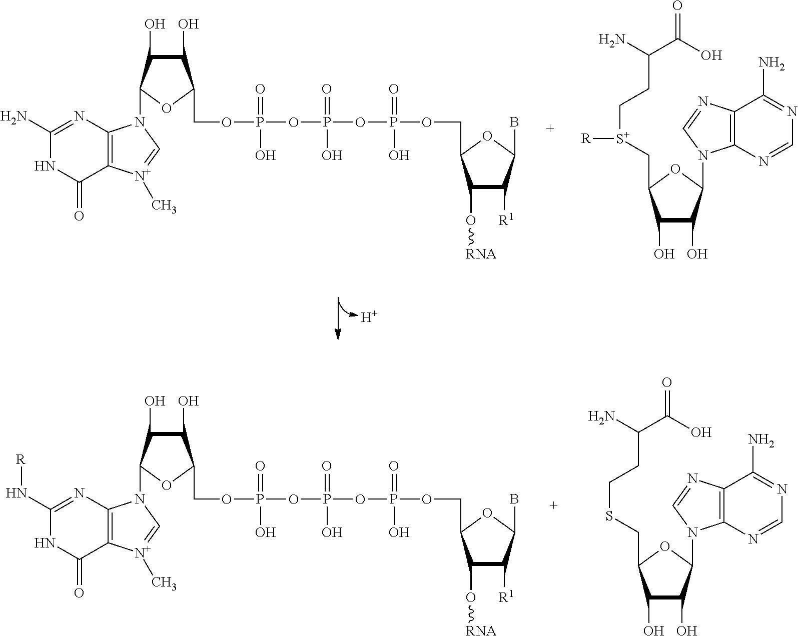

In the case in which the protein of the invention is composed of or comprises only a partial sequence of SEQ ID NO: 2, particularly preferably, the protein catalyses at least one of the above transfer reactions.

Preferably, the protein of the invention:

a. is composed of or comprises an amino acid sequence in accordance with SEQ ID NO: 3, 4, 5, 6, 7, 8, 9 or 10, or
b. is composed of or comprises an amino acid sequence which is homologous with the amino acid sequence in accordance with SEQ ID NO: 3, 4, 5, 6, 7, 8, 9 or 10, with the proviso that, in the homologous amino acid sequence, the amino acid at the position which corresponds to position 34 of SEQ ID NO: 3, 4, 5, 6, 7, 8, 9 or 10 is not valine, or
c. is composed of or comprises an amino acid sequence which is homologous with the amino acid sequence in accordance with SEQ ID NO: 3, 4, 5, 6, 7, 8, 9 or 10, wherein the amino acid sequence has more than 85%, preferably at least 90%, particularly preferably at least 95% identity with the amino acid sequence in accordance with SEQ ID NO: 3, 4, 5, 6, 7, 8, 9 or 10, with the proviso that, in the amino acid sequence, the amino acid at the position which corresponds to position 34 of SEQ ID NO: 3, 4, 5, 6, 7, 8, 9 or 10, is not valine, or
d. is composed of or comprises a contiguous partial sequence of at least 10, 15, 20, 25, 30, 35, 40, 45, 50, 60, 65, 70, 80, 90 or at least 100 amino acids, preferably at least 110, 120, 130, 140, 150, 160, 170, 180, 190 or at least 200 amino acids, particularly preferably at least 210, 220, 230, 240 or at least 250 amino acids of the amino acid sequence of a, b or c, with the proviso that the partial sequence comprises the amino at position 34 of SEQ ID NO: 3, 4, 5, 6, 7, 8, 9 or 10 or the corresponding homologous amino acid, and
e. is not composed of the amino acid sequence in accordance with SEQ ID NO: 11.

Preferably, isoleucine or threonine is not at position 34 in the amino acid sequence in accordance with SEQ ID NO: 2. Particularly preferably, alanine, glycine or methionine, preferably alanine, is at position 34 of the amino acid sequence in accordance with SEQ ID NO: 2. Preferably, aspartic acid, threonine, leucine or valine, particularly preferably aspartic acid, is at position 76 of the amino acid sequence of SEQ ID NO: 2, and/or arginine or alanine is preferably at position 92 of the amino acid sequence of SEQ ID NO: 2. Particularly preferably, alanine is at position 34 of SEQ ID NO: 2, aspartic acid is at position 76 of SEQ ID NO: 2 and arginine or alanine is at position 92 of the amino acid sequence of SEQ ID NO: 2.

The residue R is preferably propenyl CH₂═CH—CH₂—, propynyl CH≡C—CH₂—, butynyl CH≡C—CH₂—CH₂—, pentenynyl CH≡C—CH═CH—CH₂—, benzyl Ph-CH₂— or azidobutenyl N₃—CH₂—CH═CH—CH₂—, particularly preferably propenyl CH₂═CH—CH₂—, benzyl Ph-CH₂— or pentenynyl CH≡C—CH═CH—CH₂—.

In a further aspect, the present invention also relates to a nucleic acid which codes for a protein in accordance with the invention.

In a still further aspect, the present invention relates to a method for modifying the m⁷GpppN cap of a RNA molecule, in particular a mRNA molecule, comprising the step of bringing a RNA molecule provided with a m⁷GpppN cap into contact with a protein in accordance with the first aspect of the invention in the presence of an AdoMet analogue having the following formula (I):

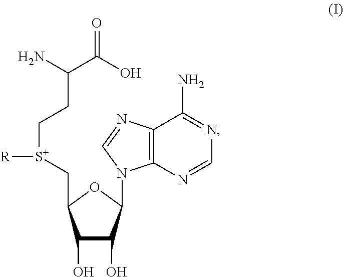

wherein R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄₋₁₀ alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted C₁₋₁₀ azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl Ph-CH₂—, propenyl CH₂═CH—CH₂—, propynyl CH≡C—CH₂—, butynyl CH≡C—CH₂—CH₂—, pentenynyl CH≡C—CH═CH—CH₂— and azidobutenyl N₃—CH₂—CH═CH—CH₂—, under conditions in which a transfer of the residue R onto the N2 of the guanosine of the m⁷GpppN cap occurs.

The residue R is preferably propenyl CH₂═CH—CH₂—, propynyl CH≡C—CH₂—, butynyl CH≡C—CH₂—CH₂—, pentenynyl CH≡C—CH═CH—CH₂—, benzyl Ph-CH₂— or azidobutenyl N₃—CH₂—CH═CH—CH₂—, particularly preferably propenyl CH₂═CH—CH₂—, benzyl Ph-CH₂— or pentenynyl CH≡C—CH═CH—CH₂—.

Particularly preferably, this method makes use of a protein in accordance with the invention which is composed of the amino acid sequence of SEQ ID NO: 2 and in which alanine, glycine or methionine, preferably alanine, is at position 34 of the amino acid sequence of SEQ ID NO: 2, aspartic acid, threonine, leucine or valine, preferably aspartic acid, is at position 76 of the amino acid sequence of SEQ ID NO: 2, and arginine or alanine is at position 92 of amino acid sequence of SEQ ID NO: 2, and AdoPropen, AdoEnYn or AdoBenzyl is used as the AdoMet analogue. More preferably, a protein in which alanine is at position 34 of SEQ ID NO: 2, aspargic acid is at position 76 of SEQ ID NO: 2 and arginine or alanine is at position 92 of the amino acid sequence of SEQ ID NO: 2, in the presence of AdoPropen, AdoEnYn or AdoBenzyl under suitable conditions, is brought into contact with the RNA, preferably mRNA, in order to allow the enzymatic reaction to occur.

Following the enzymatic modification of the m⁷GpppN cap, the residue R transferred to the N2 of the guanosine can be further modified, for example by an enzymatic or non-enzymatic chemical pathway.

In a preferred possibility, a chemical modification of the residue R is carried out using a bioorthogonal chemical reaction, preferably a bioorthogonal click reaction. Reactions of this type are known to the skilled person and include, for example, photoclick methods, thiol-ene click methods and cycloaddition reactions, for example the Cu(I)-catalyzed Huisgen cycloaddition (see, for example, H. C. Kolb, M. G. Finn, K. B. Sharpless, 2001, Angew. Chem. 113, 11, 2056-2075; H. C. Kolb, M. G. Finn, K. B. Sharpless, 2001, Angew. Chem. Int. Ed. 40, 11, 2004-2021; C. N. Bowman and C. E. Hoyle, Angew. Chem. Int. Ed. 2010, 49, 1540-1573; S. S. van Berkel, et al, Angew. Chem. 2011, 123, 8968-8989; E. Lallana et al, Angew. Chem. 2011, 123, 8956-8966; A. H. El-Sagheerab, T. Brown, Chem. Soc. Rev. 2010, 39, 1388-1405; C. S. McKay, et al, Chem. Commun. 2010, 46, 931-933; W. Song, et al, J. Am. Chem. Soc. 2008, 130, 9654-9655; P. M. E. Gramlich et al, Angew. Chem. Int. Ed. 2008, 47, 8350-8358; C. R. Becer et al, Angew. Chem. Int. Ed. 2009, 48, 4900-4908; T. R. Chan, et al, Org. Lett. 2004, 6, 2853-2855; C. Uttamapinant, et al, Angew. Chem. Int. Ed. 2012, 51, 5852-5856; Y. Wang, et al, Angew. Chem. Int. Ed. 2009, 48, 5330-5333).

The term “photoclick reaction” should be understood to mean, for example, the 1,3-dipolar cycloaddition of an alkene with a nitrile imine with the formation of a pyrazoline cycloadduct. The fundamental prerequisites for the formation of the cycloadduct herein are similar frontier orbital energies for the educts employed. In this manner, for the photoclick reaction it can be shown that the reactivity is influenced by changing the energy of the highest occupied orbital in the nitrile imine, and thus can be classified as cycloaddition type I (see Y. Wang, W. Song, W. J. Hu, Q. Lin, Angew. Chem. Int. Ed. Engl. 2009, 48, 5330-3). The photoclick reaction is not distinguished by bioorthogonality, but rather by the possibility of forming fluorescent products from non-fluorescing educts, which is of particular advantage as it avoids background signals when applied in living cells. Indications in this regard as to whether a photoclick-based functionalization of the mRNA cap is possible can be obtained by the skilled person using Kohn-Sham density functional theory calculations (KS-DFT calculations, see W. Kohn, L. J. Sham, Phys. Rev. 1965, 140, A1133-1138).

With the aid of this preferred embodiment of the method of the invention, the cap structure at the 5′ end of RNAs which comprise a cap structure of this type or which can be provided with a cap structure of this type can be specifically modified in a two-step or even multi-step method by a chemo-enzymatic pathway. In the first step, the cap structure is enzymatically modified with the aid of a protein in accordance with the invention in which, instead of the methyl residue, a residue R is transferred to the N2 of the m⁷GpppN cap. In a second chemical step, the RNA modified with this residue can then be transformed with appropriate molecules, for example with a suitable biomarker (for example biotin), for example using known click chemistry methods and further modified. Column materials also fall within this category of molecules; they allow RNAs to be immobilized via the cap structure. This immobilization can, for example, be direct or indirect via non-covalent interactions with an appropriate matrix. However, it may also occur via a covalent linkage, whereupon the interaction with the matrix is more stable allowing, for example, for more stringent washing steps which allow other components to be separated more efficiently. Thus, mRNA can, for example, be specifically isolated from complex cell lysates.

In a yet still further aspect, the present invention concerns a test kit comprising a protein in accordance with the first aspect of the invention and an AdoMet analogue in accordance with the following formula:

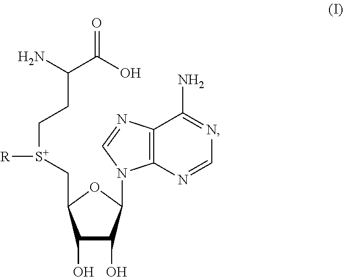

in which R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄₋₁₀ alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted C₁₋₁₀ azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl Ph-CH₂—, propenyl CH₂═CH—CH₂—, propynyl CH≡C—CH₂—, butynyl CH≡C—CH₂—CH₂—, pentenynyl CH≡C—CH═CH—CH₂— and azidobutenyl N₃—CH₂—CH═CH—CH₂—.

In a particularly preferred embodiment of the test kit in accordance with the invention, the AdoMet analogue is AdoPropen, AdoEnYn or AdoBenzyl, the protein comprises an amino acid sequence in accordance with SEQ ID NO: 2 and has alanine at position 34 of SEQ ID NO: 2, aspartic acid at position 76 of SEQ ID NO: 2 and arginine or alanine at position 92 of the amino acid sequence of SEQ ID NO: 2.

The invention will now be explained in more detail with the aid of exemplary embodiments which are provided solely for the purposes of illustration.

### 1. Synthesis of S-adenosyl-L-methionine analogues

### 1.1 Synthesis of 5′-[(S)-[(3S)-3-amino-3-carboxypropyl]prop-2-enylsulphonio]-5′-deoxyadenosine (AdoPropen)

The AdoMet analogue AdoPropen was prepared using the method described by Dalhoff et al. (C. Dalhoff et al, Nat. Chem. Biol. 2006, 2, 31-32). For the synthesis of AdoPropen, 20 mg of S-adenosyl-L-homocysteine (52 μmol) was dissolved in 3 mL of 1:1 formic acid:acetic acid, with stirring. The solution was cooled by stirring in an ice bath for 10 minutes before 264 μL (3.12 mmol) of 3-bromopropene was added. Next, the reaction solution was stirred at room temperature for 4 days and stopped by adding 30 mL of cold double-demineralized water. The aqueous phase was extracted 3 times with 5 mL of diethyl ether respectively and then the water was removed under reduced pressure. The solid obtained was dissolved in 5 mL of double-demineralized water+0.01% TFA and purified by RP-HPLC.

### 1.2 Synthesis of 5′-[(S)-[(3S)-3-amino-3-carboxypropyl]pent-2-en-4-ynylsulphonio]-5′-deoxyadenosine (AdoEnYn)

The AdoMet analogue AdoEnYn was prepared using the method described by Peters et al. (W. Peters, S. Willnow, M. Duisken, H. Kleine, T. Macherey, K. E. Duncan, D. W. Litchfield, B. Luscher, E. Weinhold, Angew Chem Int Ed Engl 2010, 49, 5170).

In order to synthesise the AdoMet analogue AdoEnYn, in a first step, pent-2-en-4-yn-1-ol was transformed into the methanesulphonic acid ester. Next, 240 mg (6.00 mmol) of sodium hydroxide was re-suspended in 6 mL of dichloromethane, 426 μL (5.50 mmol) of methanesulphonyl chloride was added and the suspension was cooled in an ice bath. Next, a mixture of (E)- and (Z)-pent-2-en-4-yn-1-ol (472 μL, 6.02 mmol) was added and the reaction mixture was stirred at room temperature for 16 hours. Extraction with 50 mL of saturated sodium bicarbonate solution was then carried out. The solvent was removed under vacuum and the activated alcohol was dissolved directly in 1 mL of a solution of methanoic acid and ethanoic acid (1:1). 7.2 mg (19 μmol) of S-adenosyl-L-homocysteine was added to this solution and it was stirred for 14 hours at room temperature. It was then placed in 30 mL of d-d H₂O and extracted three times with 50 mL of diethyl ether. The aqueous phase was frozen and freeze-dried. The residue was taken up in 2.5 mL of d-d H₂O plus 0.01% TFA and analysed using HR-ESI-MS. Purification using preparative HPLC was then carried out.

### 1.3 Synthesis of 5′-[(S)-[(3S)-3-amino-3-carboxypropyl]benzyl]-5′-deoxyadenosine (AdoBenzyl)

The AdoMet analogue AdoBenzyl was prepared using the method described by Dalhoff et al. (C. Dalhoff et al, Nat. Chem. Biol. 2006, 2, 31-32). For the synthesis of AdoBenzyl, 9.2 mg of S-adenosyl-L-homocysteine (24 μιmol) was dissolved in 1.38 mL of 1:1 formic acid:acetic acid, with stirring. The solution was cooled in an ice bath by stirring for 10 minutes before 171.2 pt (1.44 mmol) of benzyl bromide was added. Next, the reaction solution was stirred at room temperature for 4 days and stopped by adding 15 mL of cold double-demineralized water. The aqueous phase was extracted 3 times with 2.5 mL of diethyl ether respectively and then the water was removed under reduced pressure. The solid obtained was dissolved in 2.5 mL of double-demineralized water+0.01% TFA and purified by RP-HPLC.

### 2. HPLC-Coupled Activity Assay

The transfer of the propenyl (AdoPropen), pentenynyl (AdoEnYn) and benzyl (AdoBenzyl) residues carried by the synthetically produced AdoMet analogues onto the N2 atom of the guanosine of m⁷GpppA (A=adenine) enzymatically catalysed by the WT-GlaTgs2 (SEQ ID NO: 1) and the proteins of the invention in accordance with SEQ ID NOs: 4-10 was investigated with the aid of a HPLC-coupled activity assay. A typical batch with a volume of 8 μL is summarized in Table 1.

TABLE 1
Components used for the activity assay using m⁷GpppA

| | | | Final | |
|---|---|---|---|---|
| Component | Volume | | concentration | |
| GlaTgs2 or GlaTgs2 variant | 5.6 | μL | 15-50 | μM |
| MTAN/LuxS (1:1) | 0.16 | μL | 4.1 μM or 3.0 μM | |
| m⁷GpppA [10 mM] | 0.22 | μL | 275 | μM |
| AdoMet analogue | 0.33-0.6 | μL | 365-740 | μM |
| Reaction buffer 2 | qs 8 | μL | | |

Reaction buffer 2: 50 mM Tris; 10 mM MgCl₂; 100 mM NH₄OAc; pH 8.4

MTAN stands for 5′-methylthioadenosine/S-adenosylhomocysteine nucleosidase. LuxS stands for S-ribosylhomocysteine lyase.

The quantities were adjusted in proportion for larger samples of 10 or 20 μL. The quantity of AdoMet analogue used was matched such that the area of the peak in the chromatogram for a diastereoisomer of the AdoMet analogue corresponded to the area of the signal caused by m⁷GpppA. The reaction was in general stopped directly after starting the reaction (t0) and after three hours at 37° C. (t180) and examined using analytical HPLC as well as MALDI-TOF.

Table 2 below provides the activity of the GlaTgs2-WT compared with the GlaTgs2 variants, wherein AdoPropen was used as the AdoMet analogue.

TABLE 2
Activity of GlaTgs2-WT and GlaTgs2 variants on AdoPropen

| Enzyme | SEQ ID NO: | Activity | TTN |
|---|---|---|---|
| GlaTgs2-WT | 1 | + | 3 ± 2 |
| GlaTgs2-V34A | 4 | +++ | 10 ± 2 |
| GlaTgs2-V34G | 5 | ++ | 5 |
| GlaTgs2-V34M | 6 | ++ | 6 |
| GlaTgs2-V34A, D76L | 7 | + | 1 |
| GlaTgs2-V34A, D76T | 8 | + | 2 |
| GlaTgs2-V34A, D76V | 9 | + | 1 |
| GlaTgs2-V34A, R92A | 10 | +++ | 8 |

TTN = Total Turnover Number. Amino acid exchanges are given in the manner known to the skilled person (original amino acid - position - new amino acid) using the single letter code for amino acids. As an example, V34A means that the original amino acid was valine in position 34 and was replaced by alanine.

It can be seen that the proteins of the invention exhibit an activity which is at least comparable with, or higher than that of the wild type enzyme.

For the same quantity of enzyme, for example, in the case of AdoPropen, 95% of the m⁷GpppA was transformed for the variant GlaTgs2-V34A; in the case of AdoEnYn, it was 10%. As an example, with the variant GlaTgs2-V34A, a transfer of benzyl was observed.

Enzymatic parameters for the GlaTgs2 variant with SEQ ID NO: 4 with respect to AdoPropen are shown in Table 3:

TABLE 3
Enzymatic parameters for GlaTgs2-WT and
GlaTgs2-VV34A with respect to AdoPropen

| Protein | K_{M} | k_{cat} | TTN | T₅₀ (15 min) |
|---|---|---|---|---|
| GlaTgs2-WT | 151 ± 19 μM | 0.09 ± 0.08 min⁻¹ | 3 | 39.9 ± 0.2° C. |
| GlaTgs2-V34A | 57 ± 29 μM | 0.18 ± 0.08 min⁻¹ | 10 | 40.4 ± 0.2° C. |

Compared with the wild type enzyme GlaTgs2-WT, the GlaTgs2 variant GlaTgs2-V34A (SEQ ID NO: 4) exhibits a higher affinity for AdoPropen and a higher activity with AdoPropen. The thermostability is the same as that of the wild type enzyme. The kinetic parameters for AdoMet corresponded to those of the wild type enzyme (S. Hausmann, S. Shuman, J Biol Chem 2005, 280, 32101-32106).

### 3. Thermostability

The stability of a protein characterizes its ability to tolerate denaturing influences, such as an increase in the environmental temperature, within certain limits and to maintain the native conformation. The T₅₀ value can be used as a measure of the thermostability.

$$T \frac{15}{50}$$

is the temperature at which an enzyme loses 50% of its activity after 15 minutes incubation. In order to determine the T₅₀ value, the protein to be analysed is heated for 15 minutes to different temperatures in a thermocycler while a sample of the protein is incubated on ice. Next, activity tests are carried out with the previously heated proteins. The sample on ice is used as the reference, exhibiting 100% activity. After normalizing the values,

$$T \frac{15}{50} c$$

can be obtained using a Boltzmann fit and “Origin” software.

It can be seen from Table 4 that the GlaTgs2 variants GlaTgs2-V34A (SEQ ID NO: 4), GlaTgs2-V34G (SEQ ID NO: 5) and GlaTgs2-V34M (SEQ ID NO: 6) have a similar thermostability or even a higher thermostability, i.e. greater stability to higher temperatures.

TABLE 4
Thermostability of the GlaTgs2 variants GlaTgs2-V34A, -V34M and -V34G
compared with the wild type.

| Variant | SEQ ID NO: | $Thermostability , T \frac{15}{50} [ ° C . ]$ |
|---|---|---|
| WT | 1 | 39.9 ± 0.2 |
| Val34Ala | 4 | 40.4 ± 0.2 |
| Val34Gly | 5 | 44.8 |
| Val34Met | 6 | 49.0 |

### 4. Chemical Modification of Enzymatically Modified RNA Caps Using Click Chemistry

### 4.1 Biotinylation Using Thiol-Ene Click (TEC)

800 μM of m⁷GpppA was enzymatically alkenylated to propenyl² m⁷GpppA 1 (a² m⁷GpppA; propenyl at N2 of m⁷GpppA) and the reaction was stopped by adding 1/10 volumes of 1M perchloric acid or 5 minutes incubation at 68° C.

The batches were centrifuged and biotin-thiol 2 was added for the transformation. To this end, degassing was carried out for approximately 30 seconds with argon and with the exclusion of air, 1 mM of radical starter VA-044 as well as biotin thiol 2 (approximately 50 times molar excess) were added. The batches were incubated for 8 h at 44° C. and analysed using HPLC and MALDI-TOF-MS.

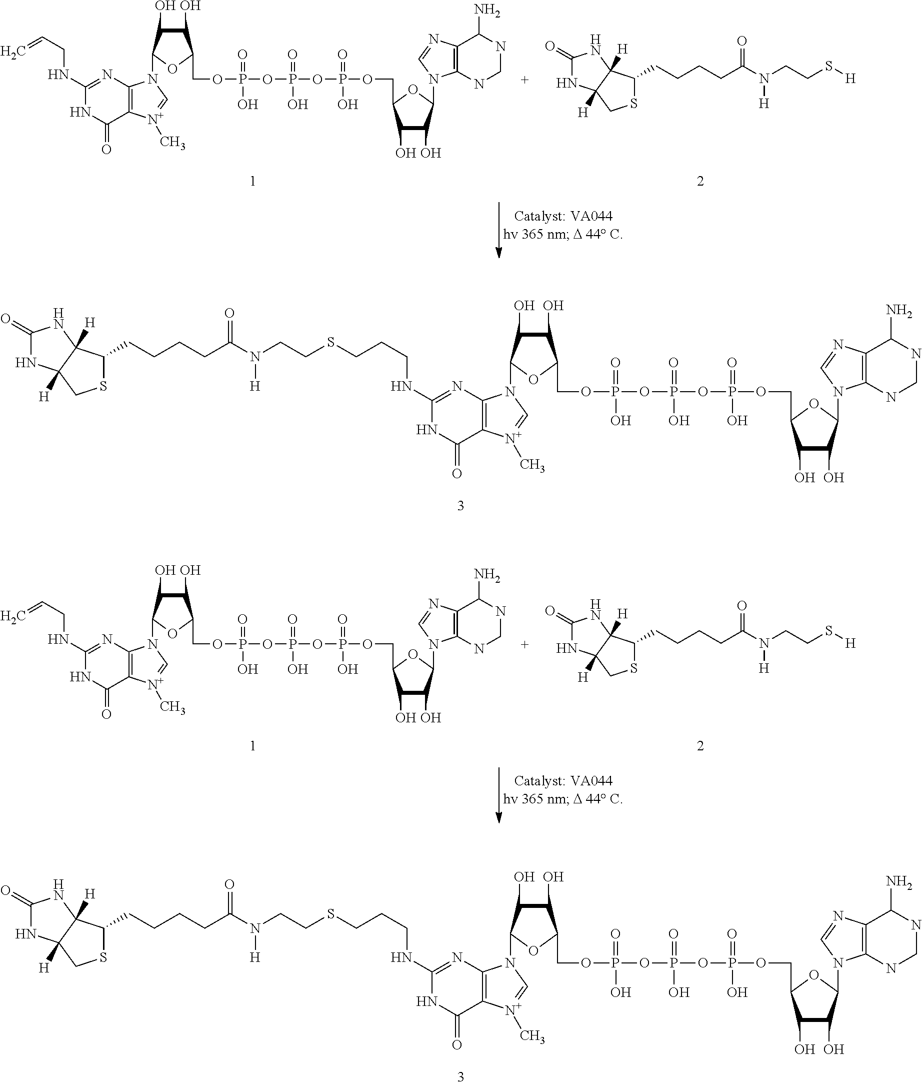

VA044=2,2′-azobis[2-(2-imidazolin-2-yl)propane]dihydrochloride

### 4.2 Fluorescence Labelling Using the Cu-Click Reaction

The Cu-click reaction was carried out using the method of Tomkuviene et al. (M. Tomkuviene, B. Clouet-d′Orval, I. Cerniauskas, E. Weinhold, S. Klimasauskas, Programmable sequence-specific click-labeling of RNA using archaeal box C/D RNP methyltransferases, Nucleic Acids Research, 2012, 40, 14, 6765). To this end, initially, approximately 100 μM of pent-2-en-4-ynyl² m⁷GpppA (p² m⁷GpppA or EnYn² m⁷GpppA) 4 was produced enzymatically and the reaction was stopped by adding 1/10 volume of 1M perchloric acid.

For the Cu-click reaction, 300 mM of CuBr solution (in DMSO/tBuOH 3:1) was freshly prepared and diluted by 1:10 with 111 mM of TBTA solution (in DMSO/tBuOH 3:1).

Next, 8 μL of DMSO/tBuOH, 3 μL of the 30 mM CuBr solution (in TBTA) as well as 2.5 μL of Eterneon azide 5 (Eterneon azide 480/635, Jena Bioscience GmbH, Cat. No. CLK-FA15-1) (2.5 mM in DMSO/tBuOH) were added. The reaction was incubated at 37° C. for one hour with occasional vortexing and analysed by gel electrophoresis. TBTA=tris[(1-benzyl-1H-1,2,3-triazol-4-yl)methyl]amine), DMSO=dimethylsulphoxide.

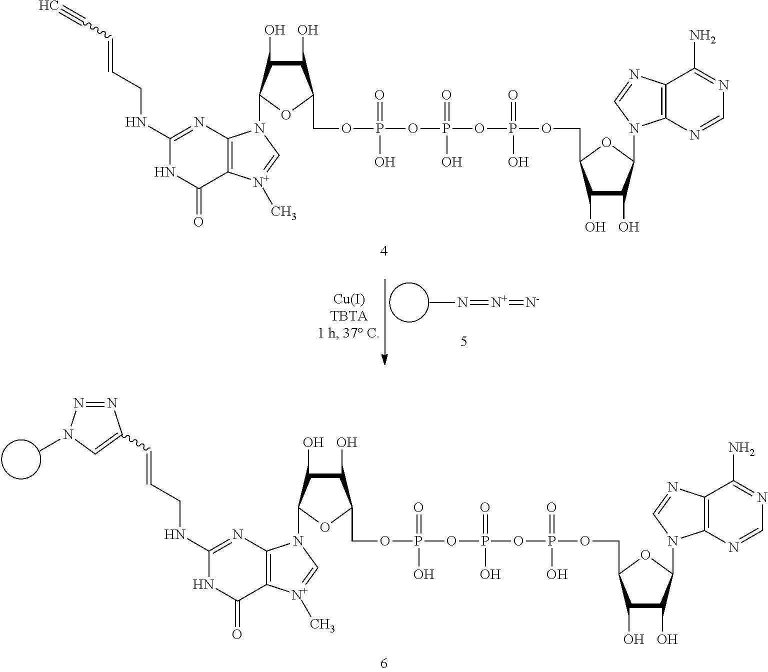

### 4.3 Fluorescence Labelling by Cross Metathesis

Fluorescence labelling of allyl-modified mRNA caps was carried out using cross metathesis in accordance with the general scheme given below using fluorescein o-acrylate 8 or another fluorescence-labelled acrylate and with the aid of a 2^{nd} generation Hoveyda-Grubbs catalyst 10:

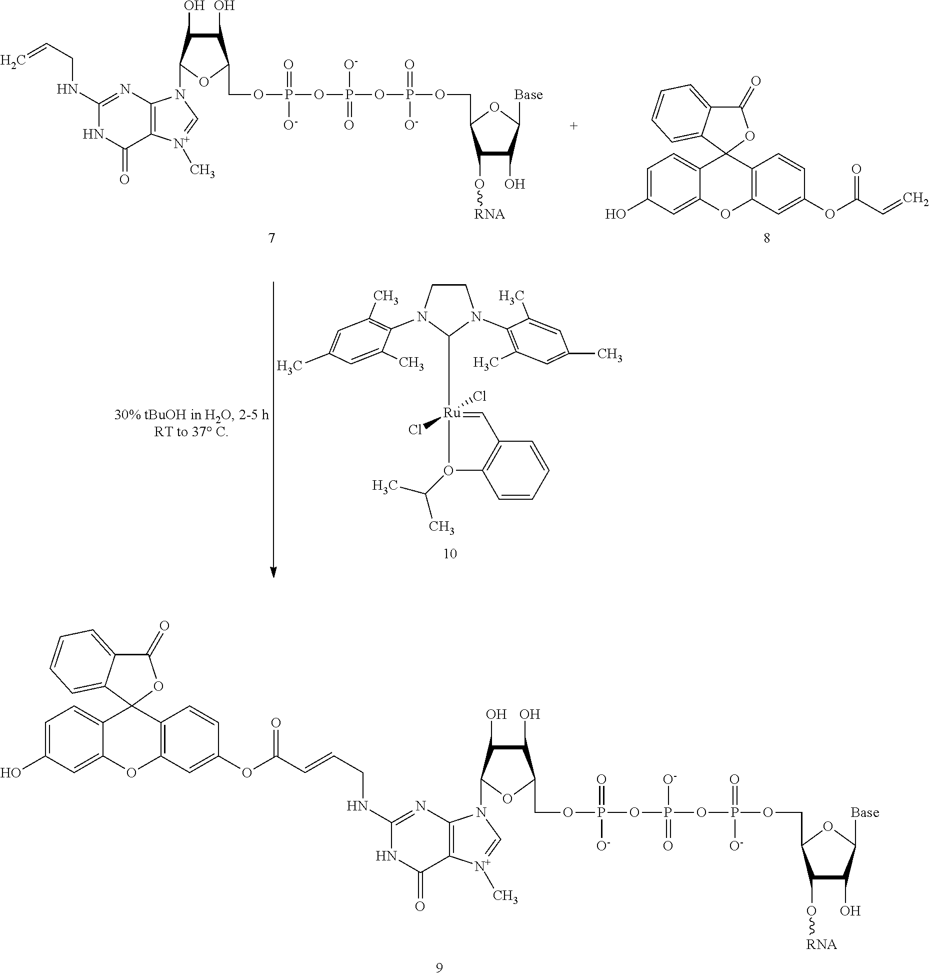

To this end, as an example, the allyl-modified mRNA cap 7 was incubated with two equivalents of the fluorescein-o-acrylate 8, which had been dissolved in DMSO, as well as 2 mol % of the Hoveyda-Grubbs catalyst 10 which had been taken up in 30% tBuOH in water, also in 30% tBuOH in water for five hours at room temperature or 37° C. with the exclusion of light. The analysis was carried out using 20% polyacrylamide gel electrophoresis and in-gel fluorescence detection.

### 4.4 Functionalization of Cap Structures Using Photoclick Reaction

As an example, fluorescence labelling of an alkenylated and an alkynylated mRNA cap using the photoclick reaction will now be described. To this end, the nitrile amines 11a and 12a of the tetrazoles 11 and 12 were transformed with the cap analogue m⁷GpppA.

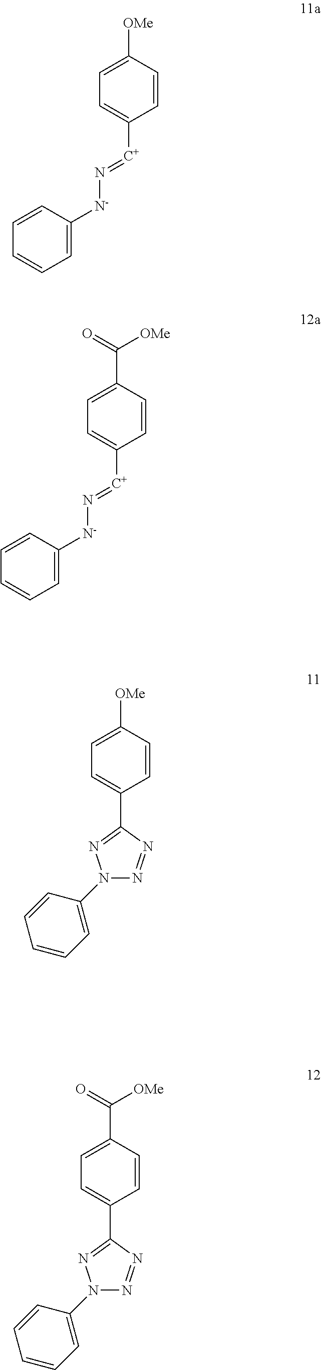 [SMILES: COc1ccc(/[C+]=N/[N-]c2ccccc2)cc1 ; COC(=O)c1ccc(/[C+]=N/[N-]c2ccccc2)cc1 ; COc1ccc(-c2nnn(-c3ccccc3)n2)cc1 ; COC(=O)c1ccc(-c2nnn(-c3ccccc3)n2)cc1]

### 4.4.1 Fluorescence Labelling of an Alkenylated mRNA Cap

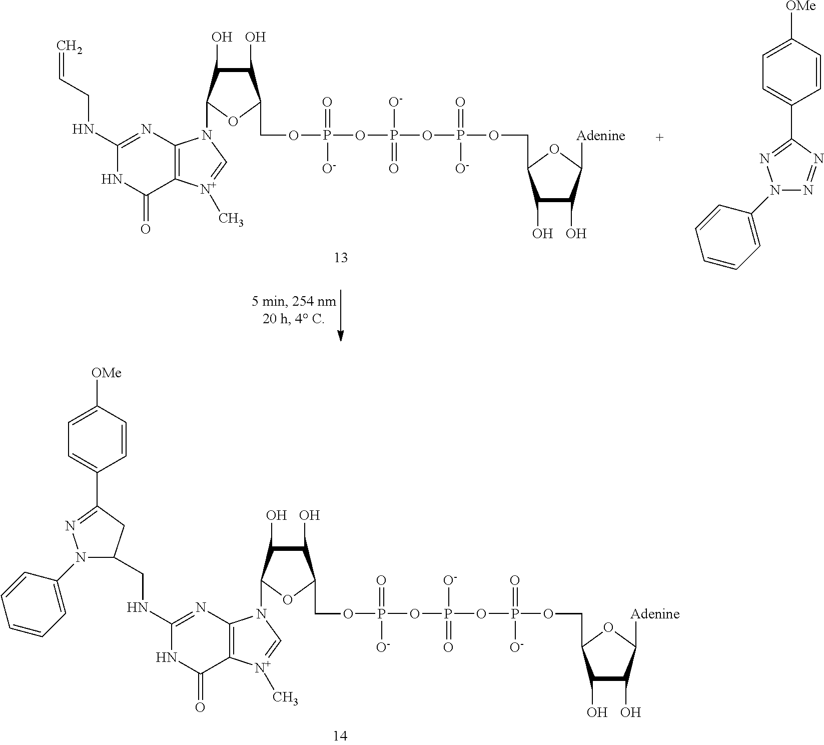

Firstly, 1 mM of m⁷GpppA was reacted in the presence of AdoPropen and GlaTgs2-V34A to form a² m⁷GpppA and the resulting mixture was then heated to 68° C. and dialyzed in order to remove unreacted AdoPropen. The removal of unreacted AdoPropen served to prevent it from also reacting with the nitrile imine in the following photoclick reaction. After these steps, the aqueous solution of a² m⁷GpppA was supplemented with acetonitrile and with tetrazole 11 (617 μM) and after careful mixing, it was irradiated in a black mictotitre plate for five minutes at 254 nm. It was shown that irradiating at a minimum distance from the reaction and light source was essential to successful performance and the formation of the reactive nitrile imine when the distance was increased did not appear to happen, or was minimized. The components contained in the sample were separated by gel electrophoresis after incubating for up to 20 hours at 4° C. and analysed as to the occurrence of fluorescent products. To this end, the gel was irradiated at a wavelength of 365 nm and photographed. For the reaction which was carried out in the presence of the alkenylated a² m⁷GpppA cap, a turquoise fluorescent product could be detected. An analysis of the gel by UV shadowing carried out at the same time showed that the detected fluorescent product had a lower electromobility and thus presumably a higher molecular weight than the cap. This was in agreement with the possibility that the band in question was the expected photoclick product (P¹-adenosin(5′)-P³—[N²-ethyl-2-(4-(4-methoxyphenyl)-2-phenylpyrazoline), 7-methylguanosine(5′)]triphosphate; synonym: N²-methoxypyrazolinethyl-m⁷GpppA) 14, since this would have a higher molecular weight than the cap analogue a² m⁷GpppA 13 for the same charge. In control experiments, a corresponding fluorescing signal was detected. The controls involved carrying out the bioconversion without an enzyme, without AdoPropen, without m⁷GpppA and in the presence of denatured enzyme. In all of these controls, then, no alkenylated cap which could form the substrate for the photoclick reaction was shown not to have formed. Since, then, in the absence of a² m⁷GpppA no fluorescent product in accordance with the photoclick reaction could be observed, this means that fluorescence labelling of the alkenylated cap with tetrazole 11 could be carried out successfully and specifically.

This was also verified by mass spectrometric analysis using HPLC-ESI-TOF-MS. In this regard, the mass of the expected photoclick product 14 was detected in the reaction mixture (reported [M]⁺=1051.23 m/z; determined [M]⁺=1051.23 m/z).

In order to further characterize the photoclick reaction of tetrazole 11 and a² m⁷GpppA 13 as regards potential applications in cells, a first kinetic analysis was carried out. To this end, the reaction as described above was carried out, but a respective portion of the sample was analysed immediately after incubation of 5, 30, 120 and 240 minutes by detection of fluorescent products. This showed that the photoclick product 14 could already be detected after 5 minutes, whereas at later times, no further transformation was observed on the basis of the detected fluorescence intensity. This means that because of its kinetics, the reaction was also suitable for visualizing mRNA in living cells, since a visible signal can be observed even a short period after induction. The investigation of PC3 cells after a five minute irradiation with UV light (λ=254 nm) also showed that an effect on the cell morphology but not on its vitality could be observed, so that an application without killing the cells during the investigation by the UV irradiation is possible. In addition to forming a fluorescent product using a² m⁷GpppA as the educt and the high reaction rate, then, the duration of the irradiation in order to activate the tetrazole was compatible with applications in living cells.

The photoclick reactions described above were also carried out successfully for the combination of nitrile imine 12a and a² m⁷GpppA 13, and the corresponding photoclick product (P¹-adenosine(5′)-P³—[N²-ethyl-2-(4-(4-methylbenzoate)-2-phenylpyrazoline), 7-methylguanosine(5′)]triphosphate; synonym: N²-benzoatepyrazoline ethyl m⁷GpppA) 16 was obtained.

In this regard it was observed that the emission maxima of the photoclick products of the tetrazoles 11 and 12 with a² m⁷GpppA appear to differ. The photoclick reactions can thus also be used under some circumstances in order to produce a fluorophore emitting at any wavelength even in vivo.

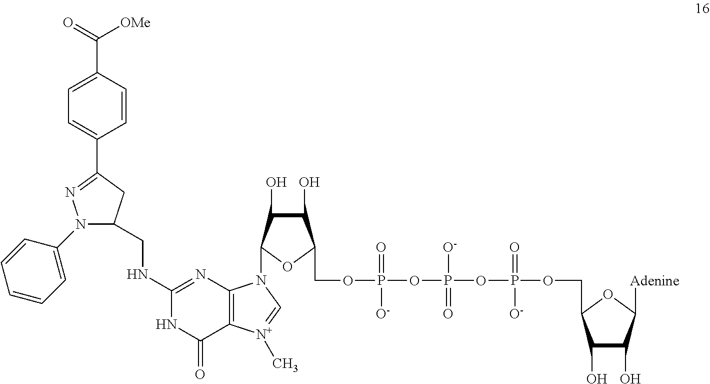

### 4.4.2 Fluorescence Labelling of an Alkynylated mRNA Cap

A photoclick reaction for the combination of nitrile imine 11a and p² m⁷GpppA 15 was also carried out.

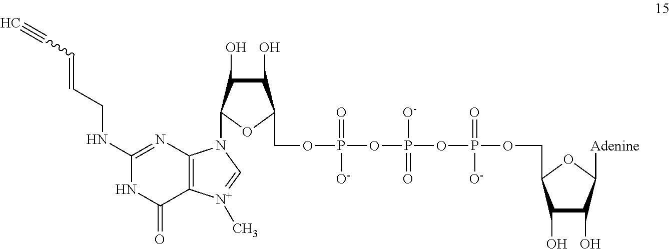

To this end, the photoclick reaction described above was carried out to fluorescence-label a² m⁷GpppA using tetrazole 11 in order to modify the alkynylated cap analogue p² m⁷GpppA 15. The bioconversion of m⁷GpppA with AdoEnYn was thus carried out with GlaTgs2-V34A and as a control in the presence of the denatured enzyme. This ensured that the same components were present in both batches, but in the control the formation of the photoclick educt p² m⁷GpppA did not occur. After successful initiation of the photoclick reaction by irradiation at a wavelength of 254 nm, the reaction and control batches were incubated for 20 hours at 4° C. and after separation of the components obtained were separated by gel electrophoresis in order to form a fluorescent cycloadduct. By illuminating the gel at a wavelength of 365 nm, a fluorescent band could be detected in the reaction mixture which did not appear in the control and thus could be assigned to the corresponding pyrazoline (P¹-adenosin(5′)-P³—[N²-but-2-en-4-(4-(4-methoxyphenyl)-2-phenylpyrazolin)yl, 7-methylguanosine(5′)]triphosphate; synonym: N²-methoxypyrazolin butenyl-m⁷GpppA) 17.

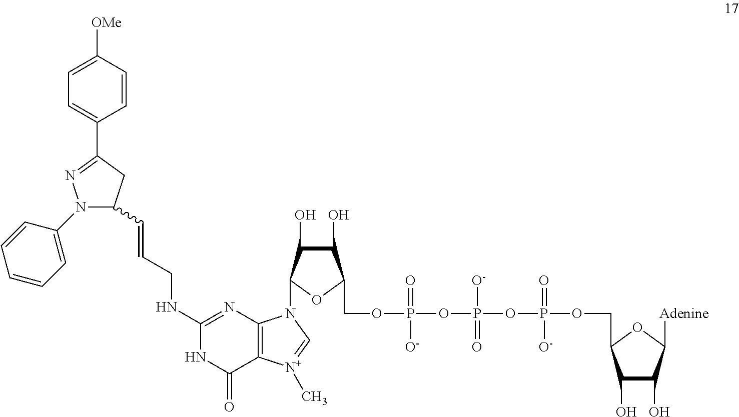

1. An isolated or synthetic protein which:
a. is composed of or comprises an amino acid sequence in accordance with SEQ ID NO: 2, or
b. is composed of or comprises an amino acid sequence which is homologous with the amino acid sequence in accordance with SEQ ID NO: 2, with the proviso that, in the homologous amino acid sequence, the amino acid at the position which corresponds to position 34 of SEQ ID NO: 2 is not valine, or
c. is composed of or comprises an amino acid sequence which is homologous with the amino acid sequence in accordance with SEQ ID NO: 2, wherein the amino acid sequence has more than 85% identity with the amino acid sequence in accordance with SEQ ID NO: 2, with the proviso that, in the homologous amino acid sequence, the amino acid at the position which corresponds to position 34 of SEQ ID NO: 2 is not valine, or
d. is composed of or comprises a contiguous partial sequence of at least 10 amino acids of the amino acid sequence of a, b or c, with the proviso that the partial sequence comprises the amino acid at position 34 of SEQ ID NO: 2 or the corresponding homologous amino acid, and
e. is not composed of the amino acid sequence in accordance with SEQ ID NO: 11.

2. The protein as claimed in claim 1, wherein the protein enzymatically catalyses the transfer of the residue R of the compound with general formula (I): 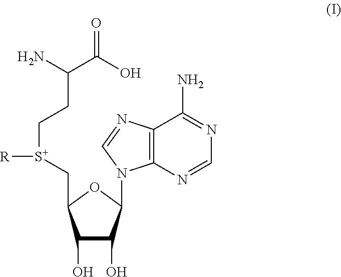
to the N2 of the guanosine of m⁷GTP, m⁷GpppN or a compound with the following formula (II): 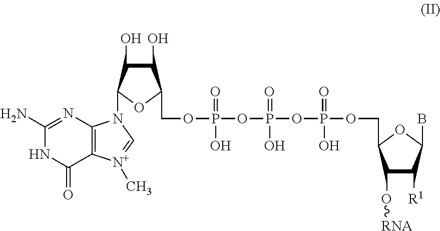
and wherein RNA means ribonucleic acid, R¹ means OH or OCH₃, N means nucleoside, nucleotide, nucleoside or nucleotide analogue, B stands for nucleobase, and R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄₋₁₀ alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl, propenyl CH₂═CH—CH₂—, propynyl CH≡C—CH₂—, butynyl CH≡C—CH₂—CH₂—, pentenynyl CH≡C—CH═CH—CH₂—, and azidobutenyl N₃—CH₂—CH═CH—CH₂—.

3. The protein as claimed in claim 1, wherein the protein:
a. is composed of or comprises an amino acid sequence in accordance with SEQ ID NO: 3, 4, 5, 6, 7, 8, 9 or 10, or
b. is composed of or comprises an amino acid sequence which is homologous with the amino acid sequence in accordance with SEQ ID NO: 3, 4, 5, 6, 7, 8, 9 or 10, with the proviso that in the homologous amino acid sequence the amino acid at the position which corresponds to position 34 of SEQ ID NO: 3, 4, 5, 6, 7, 8, 9 or 10 is not valine, or
c. is composed of or comprises an amino acid sequence which is homologous with the amino acid sequence in accordance with SEQ ID NO: 3, 4, 5, 6, 7, 8, 9 or 10, wherein the amino acid sequence has more than 85% identity with the amino acid sequence in accordance with SEQ ID NO: 3, 4, 5, 6, 7, 8, 9 or 10, with the proviso that in the homologous amino acid sequence the amino acid at the position which corresponds to position 34 of SEQ ID NO: 3, 4, 5, 6, 7, 8, 9 or 10 is not valine, or
d. is composed of or comprises a contiguous partial sequence of at least 10 amino acids of the amino acid sequence of a, b or c, with the proviso that the partial sequence comprises the amino acid at position 34 of SEQ ID NO: 3, 4, 5, 6, 7, 8, 9 or 10 or the corresponding homologous amino acid, and
e. is not composed of the amino acid sequence in accordance with SEQ ID NO: 11.

4. The protein as claimed in claim 1, wherein alanine, glycine or methionine, preferably alanine, is at position 34 of the amino acid sequence in accordance with SEQ ID NO: 2.

5. The protein as claimed in claim 1, wherein aspartic acid, threonine, leucine or valine, particularly preferably aspartic acid, is at position 76 of the amino acid sequence of SEQ ID NO: 2, and/or arginine or alanine is at position 92 of the amino acid sequence of SEQ ID NO: 2.

6. The protein as claimed in claim 2, wherein R is CH₂═CH—CH₂—, CH≡C—CH₂—, CH≡C—CH₂—CH₂—, CH≡C—CH═CH—CH₂—, Ph-CH₂— or N₃—CH₂—CH═CH—CH₂.

7. A nucleic acid which codes for a protein as claimed in claim 1.

8. A method for modifying the m⁷GpppN cap of a RNA molecule, in particular a mRNA molecule, comprising the step of bringing a RNA molecule provided with a m⁷GpppN cap into contact with a protein as claimed in one of claims 1 to 6 in the presence of an AdoMet analogue having the following formula (I): 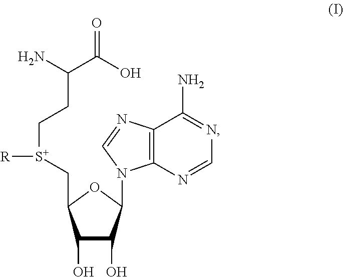
wherein R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄-10 alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted C₁₋₁₀ azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl, propenyl CH₂═CH—CH₂—, propynyl CH≡C—CH₂—, butynyl CH≡C—CH₂—CH₂—, pentenynyl CH≡C—CH═CH—CH₂— and azidobutenyl N₃—CH₂—CH═CH—CH₂—,
under conditions in which a transfer of the residue R onto the N2 of the guanosine of the m⁷GpppN cap occurs.

9. The method as claimed in claim 8, wherein the RNA, in the presence of AdoPropen or AdoEnYn, is brought into contact with a protein which comprises the amino acid sequence of SEQ ID NO: 2 and in which alanine is at position 34 of SEQ ID NO: 2, aspartic acid is at position 76 of SEQ ID NO: 2 and arginine or alanine is at position 92 of the amino acid sequence of SEQ ID NO: 2.

10. The method as claimed in claim 8, comprising the further step of subsequent chemical modification of the residue R transferred to the N2 of the guanosine of the m⁷GpppN cap.

11. The method as claimed in claim 10, wherein the chemical modification of the residue R is carried out by means of a photoclick reaction.

12. A test kit comprising a protein in accordance with claim 1 and an AdoMet analogue in accordance with the following formula (I): 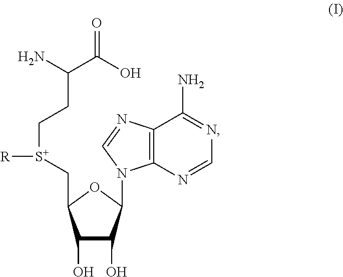
in which R is selected from the group consisting of substituted or unsubstituted C₂₋₁₀ alkyl, substituted or unsubstituted C₂₋₁₀ alkenyl, substituted or unsubstituted C₂₋₁₀ alkynyl, substituted or unsubstituted C₄₋₁₀ alkenynyl, substituted or unsubstituted C₃₋₁₂ cycloalkyl, substituted or unsubstituted C₃₋₁₂ cycloalkenyl, substituted or unsubstituted C₅₋₁₂ cycloalkynyl, substituted or unsubstituted C₅₋₁₂ cycloalkenynyl, substituted or unsubstituted C₁₋₁₀ heteroalkyl, substituted or unsubstituted C₂₋₁₀ heteroalkenyl, substituted or unsubstituted C₂₋₁₀ heteroalkynyl, substituted or unsubstituted C₄₋₁₀ heteroalkenynyl, substituted or unsubstituted C₁₋₁₀ azidoalkyl, substituted or unsubstituted C₂₋₁₀ azidoalkenyl, substituted or unsubstituted C₂₋₁₀ azidoalkynyl, substituted or unsubstituted C₄₋₁₀ azidoalkenynyl, substituted or unsubstituted benzyl, propenyl CH₂═CH—CH₂—, propynyl CH≡C—CH₂—, butynyl CH≡C—CH₂—CH₂—, pentenynyl CH≡C—CH═CH—CH₂— and azidobutenyl N₃—CH₂—CH═CH—CH₂—.

13. The test kit as claimed in claim 12, wherein the AdoMet analogue is AdoPropen, AdoEnYn or AdoBenzyl, the protein is composed of an amino acid sequence in accordance with SEQ ID NO: 2 and the protein has alanine at position 34 of SEQ ID NO: 2, aspartic acid at position 76 of SEQ ID NO: 2 and arginine or alanine at position 92 of the amino acid sequence of SEQ ID NO: 2.

14. The protein as claimed in claim 1, wherein the protein:
c. is composed of or comprises an amino acid sequence which is homologous with the amino acid sequence in accordance with SEQ ID NO: 2, wherein the amino acid sequence has at least 90% identity with the amino acid sequence in accordance with SEQ ID NO: 2, with the proviso that, in the homologous amino acid sequence, the amino acid at the position which corresponds to position 34 of SEQ ID NO: 2 is not valine, or
d. is composed of or comprises a contiguous partial sequence of at least 30 amino acids of the amino acid sequence of a, b or c.

15. The protein as claimed in claim 1, wherein the protein:
c. is composed of or comprises an amino acid sequence which is homologous with the amino acid sequence in accordance with SEQ ID NO: 2, wherein the amino acid sequence has at least 95% identity with the amino acid sequence in accordance with SEQ ID NO: 2, with the proviso that, in the homologous amino acid sequence, the amino acid at the position which corresponds to position 34 of SEQ ID NO: 2 is not valine, or
d. is composed of or comprises a contiguous partial sequence of at least 110 amino acids of the amino acid sequence of a, b or c, with the proviso that the partial sequence comprises the amino acid at position 34 of SEQ ID NO: 2 or the corresponding homologous amino acid.

16. The protein as claimed in claim 1, wherein the protein:
d. is composed of or comprises a contiguous partial sequence of at least 210 amino acids of the amino acid sequence of a, b or c.

17. The protein as claimed in claim 3, wherein the protein:
c. is composed of or comprises an amino acid sequence which is homologous with the amino acid sequence in accordance with SEQ ID NO: 3, 4, 5, 6, 7, 8, 9 or 10, wherein the amino acid sequence has more than 90% identity with the amino acid sequence in accordance with SEQ ID NO: 3, 4, 5, 6, 7, 8, 9 or 10, with the proviso that in the homologous amino acid sequence the amino acid at the position which corresponds to position 34 of SEQ ID NO: 3, 4, 5, 6, 7, 8, 9 or 10 is not valine, or
d. is composed of or comprises a contiguous partial sequence of at least 30 amino acids of the amino acid sequence of a, b or c.

18. The protein as claimed in claim 3, wherein the protein:
c. is composed of or comprises an amino acid sequence which is homologous with the amino acid sequence in accordance with SEQ ID NO: 3, 4, 5, 6, 7, 8, 9 or 10, wherein the amino acid sequence has more than 95% identity with the amino acid sequence in accordance with SEQ ID NO: 3, 4, 5, 6, 7, 8, 9 or 10, with the proviso that in the homologous amino acid sequence the amino acid at the position which corresponds to position 34 of SEQ ID NO: 3, 4, 5, 6, 7, 8, 9 or 10 is not valine, or
d. is composed of or comprises a contiguous partial sequence of at least 110 amino acids of the amino acid sequence of a, b or c.

19. The protein as claimed in claim 3, wherein the protein:
d. is composed of or comprises a contiguous partial sequence of at least 210 amino acids of the amino acid sequence of a, b or c.

20. The protein as claimed in claim 2, wherein R is CH₂═CH—CH₂—, Ph-CH₂— or CH≡C—CH═CH—CH₂—.
